(19) United States
(12) Patent Application Publication
(10) Pub. No.: US 2026/0083546 A1
(43) Pub. Date: Mar. 26, 2026

(54) FOLDED STENT GRAFT AND METHOD OF MANUFACTURE

(71) Applicant: Merit Medical Systems, Inc., South Jordan, UT (US)

(72) Inventors: John Hall; Wayne Mower, Bountiful, UT (US)

(21) Appl. No.: 19/333,705

(22) Filed: Sep. 19, 2025

Related U.S. Application Data
(60) Provisional application No. 63/697,086, filed on Sep. 20, 2024.

(51) Int. Cl. A61F 2/07; A61L 31/04; A61L 31/10

(52) U.S. Cl. CPC A61F 2/07; A61L 31/048; A61L 31/10; A61F 2240/001

(57) ABSTRACT

Medical devices, including covered or coated stents and stent grafts are disclosed. In some embodiments, one or more layers of a covering may be bent, folded, inverted, everted, or otherwise disposed to cover and couple to a stent.

### RELATED APPLICATIONS

The present application claims priority to U.S. Provisional Application No. 63/697,086, filed on Sep. 20, 2024 and titled, “Folded Stent Graft and Method of Manufacture,” which is hereby incorporated by reference in its entirety.

### TECHNICAL FIELD

The present disclosure relates generally to medical devices. More specifically, the present disclosure relates to medical appliances or other prostheses, particularly those made of, constructed from, covered or coated stent grafts.

### BRIEF DESCRIPTION OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1A is a lateral, cutaway view of a covered stent, according to embodiments described herein.

FIG. 1B is a cross-sectional view of the covered stent of FIG. 1A taken through line 1B-1B, according to embodiments described herein.

FIG. 2A is a lateral, cutaway view of a covered stent, according to embodiments described herein.

FIG. 2B is a lateral, cutaway view of the covered stent of FIG. 2A, according to embodiments described herein.

FIG. 2C is a cross-sectional view of the covered stent graft 200 of FIG. 2B taken through line 2C-2C, according to embodiments described herein.

FIG. 3A is a lateral, cutaway view of a covered stent, according to embodiments described herein.

FIG. 3B is a cross-sectional view of the covered stent of FIG. 3A taken through line 3B-3B, according to embodiments described herein.

FIG. 4A is a lateral, cutaway view of a covered stent, according to embodiments described herein.

FIG. 4B is a cross-sectional view of the covered stent of FIG. 4A taken through line 4B-4B, according to embodiments described herein.

FIG. 5A is a lateral, cutaway view of a covered stent, according to embodiments described herein.

FIG. 5B is a cross-sectional view of the covered stent of FIG. 5A taken through line 5B-5B, according to embodiments described herein.

### DETAILED DESCRIPTION

Medical appliances, such as stent grafts, treat various vascular conditions by providing structural support and promoting blood flow restoration within body lumens. A stent graft is a combination of a stent, which offers mechanical support, and a graft, which serves as a conduit for blood flow. Stent grafts may, for example, be used in connection with minimally invasive solutions for managing vascular pathologies.

A stent graft is often composed of covering (e.g., a graft) including one or more bonded layers of materials, and a scaffolding structure (e.g., a stent). In some embodiments, through the application of heat, pressure, and/or adhesives, the stent, graft, and graft layers are bonded together, to form a unified structure.

In some instances, during use, deployment, or manufacture, there is a risk of delamination—the separation or splitting of layers within the stent graft structure. While delamination can manifest throughout a stent graft, it can become particularly problematic when affecting the terminal ends. Delamination at a terminal end of a stent graft can lead to a host of major complications. For instance, delamination at a terminal end can compromise the seal integrity between the graft material and the vessel wall. A compromised seal can result in an endoleak, where blood leaks into an aneurysm sac.

Further complications, which can be triggered by delamination, can include the weakening of the structural integrity of the stent graft. This can result in device migration, fracture, fragmentation, or deformation. Delamination can also increase thrombogenicity by creating irregular surfaces or pockets that impede blood flow. Moreover, bacteria can accumulated within the spaces or gaps created by delamination, increasing the risk of infection. Accordingly, delamination can accelerate device degradation, reducing device lifespan, increasing costs through remediation or device replacement, and exposing patients to additional procedural risks.

Aspects and implementations of the present disclosure address the above and other challenges by providing stent grafts and method of manufacturing that addresses delamination of a stent graft's layers at a terminal end. In embodiments, one or more graft layers can be placed concentrically external and/or internal to one or more stents. The one or more graft layers can then be everted (e.g., evaginated, folded outward, etc.), or inverted (e.g., involuted, folded inward, etc.) around the stent. The stent graft can then be bonded, sealing the one or more graft layers and one or more stents. Through such a process, the terminal ends of the overall stent graft can be formed from continuous material of the graft layers. The terminal ends of the everted or inverted graft layers can lie elsewhere (e.g., on a midbody or middle portion) of the stent graft.

Advantages of the present disclosure include introduction of a stent graft (and method of manufacture) that is less susceptible to delamination and consequently, migration, fracture, fragmentation and/or deformation. The proposed stent graft (and method of manufacture) includes enhanced durability, particularly at terminal ends of the stent graft.

As used herein, the term “stent” refers to a medical appliance configured for use within a bodily structure, such as within a body lumen. A stent can include a scaffolding or support structure, such as a frame, and/or a covering. Thus, as used herein, “stent” refers to both covered (e.g., stent grafts) and uncovered scaffolding structures.

In embodiments, the current disclosure may be applicable to stents, or other medical appliances designed for the central venous (“CV”) system, peripheral vascular (“PV”) stents, abdominal aortic aneurism (“AAA”) stents, bronchial stents, esophageal stents, biliary stents, coronary stents, gastrointestinal stents, neuro stents, thoracic aortic endographs, or any other stent or stent graft. Additionally, the present disclosure encompasses bifurcated stents, or stents with a bifurcation. For example, bifurcated stents can be designed for placement in blood vessels that bifurcate.

The present disclosure may be equally applicable to other prosthesis such as grafts. Any medical appliance comprised of materials herein described may be configured for use or implantation within various areas of the body, including vascular, cranial, thoracic, pulmonary, esophageal, abdominal, or ocular application. Examples of medical appliances within the scope of this disclosure include, but are not limited to, stents, vascular grafts, stent grafts, cardiovascular patches, reconstructive tissue patches, hernia patches, general surgical patches, heart valves, sutures, dental reconstructive tissues, medical device coverings and coatings, gastrointestinal devices, blood filters, artificial organs, ocular implants, and pulmonary devices, including pulmonary stents.

For convenience, many of the specific examples included below reference stents. Notwithstanding any of the particular medical appliances referenced in the examples or disclosure below, the disclosure and examples may apply analogously to any prostheses or other medical appliance.

It will be readily understood that the components of the embodiments as generally described and illustrated in the Figures herein could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “proximal” and “distal” are used herein to refer to opposite locations on a stent or another medical appliance. The proximal end of an appliance is defined as the end closest to the practitioner when the appliance is disposed within a deployment device which is being used by the practitioner. The distal end is the end opposite the proximal end, along the longitudinal direction of the appliance, or the end furthest from the practitioner. It is understood that, as used in the art, these terms may have different meanings once the appliance is deployed (i.e., the “proximal” end may refer to the end closest to the head or heart of the patient depending on application). For consistency, as used herein, the ends labeled “proximal” and “distal” prior to deployment remain the same regardless of whether the appliance is deployed. The longitudinal direction of a stent is the direction along the axis of a generally tubular stent.

In embodiments where a stent or another appliance is composed of a metal wire structure coupled to one or more layers of a film or sheet like components, such as a polymer layer, the metal structure is referred to as the “scaffolding” or “frame,” and the polymer layer as the “covering” or “coating.” The terms “covering” or “coating” may refer to a single layer of polymer, multiple layers of the same polymer, or layers comprising distinct polymers used in combination. Furthermore, as used herein, the terms “covering” and “coating” refer only to a layer or layers which are coupled to a portion of the scaffold; neither term requires that the entire scaffold be “covered” or “coated.” In other words, medical appliances wherein portion of the scaffold may be covered and a portion remain bare, are within the scope of this disclosure. Finally, any disclosure recited in connection with coverings or coatings may analogously be applied to medical devices comprising one or more “covering” layers with no associated frame or other structure. For example, a hernia patch comprising any of the materials described herein as “coatings” or “coverings” is within the scope of this disclosure regardless of whether the patch further comprising a frame or other structure.

Medical device coverings may comprise multilayered constructs, comprised of two or more layers which may be serially applied. Further, multilayered constructs may comprise nonhomogeneous layers, meaning adjacent layers have differing properties. Thus, as used herein, each layer of a multilayered construct may comprise a distinct layer, either due to the distinct application of the layers or due to differing properties between layers.

Additionally, as used herein, “tissue ingrowth” or “cellular penetration” refer to any presence or penetration of a biological or bodily material into a component of a medical appliance. For example, the presence of body tissues (e.g. collagen, cells, and so on) within an opening or pore of a layer or component of a medical appliance comprises tissue ingrowth into that component. Further, as used herein, “attachment” of tissue to a component of a medical appliance refers to any bonding or adherence of a tissue to the appliance, including indirect bonds. For example, tissue of some kind (e.g. collagen) may become attached to a stent covering (including attachment via tissue ingrowth) and another layer of biologic material (such as endothelial cells) may, in turn, adhere to the first tissue. In such instances, the second biologic material (endothelial cells in the example), and the tissue (collagen in the example) are “attached” to the stent covering.

Furthermore, through the present disclosure, certain fibrous materials (such as rotational spun materials) may be referred to as inhibiting or promoting certain biological responses. These relative terms are intended to reference the characteristics of the fibrous materials with respect to non-fibrous materials or coatings. Examples of non-fibrous coatings include non-fibrous PTFE sheets, other similarly formed polymers, and the like. Examples of fibrous coatings include rotational spun PTFE, electrospun PTFE, expanded PTFE, and other similarly formed polymers or materials. Examples of spun fibrous coatings include rotational spun PTFE, electrospun PTFE, and other similarly formed polymers or materials, and exclude expanded PTFE.

Lumens within the circulatory system are generally lined with a single layer (monolayer) of endothelial cells. This lining of endothelial cells makes up the endothelium. The endothelium acts as an interface between blood flowing through the lumens of the circulatory system and the inner walls of the lumens. The endothelium, among other functions, reduces or prevents turbulent blood flow within the lumen. The endothelium plays a role in many aspects of vascular biology, including atherosclerosis, creating a selective barrier around the lumen, blood clotting, inflammation, angiogenesis, vasoconstriction, and vasodilation.

A therapeutic medical appliance which includes a covering of porous or semi-porous material may permit the formation of an endothelial layer onto the porous surface of the blood contact side of the medical device. Formation of an endothelial layer on a surface, or endothelialization, may increase the biocompatibility of an implanted device. For example, a stent which permits the formation of the endothelium on the inside diameter (blood contacting surface) of the stent may further promote healing at the therapeutic region and/or have longer term viability. For example, a stent coated with endothelial cells may be more consistent with the surrounding body lumens, thereby resulting in less turbulent blood flow or a decreased risk of thrombosis, or the formation of blood clots. A stent which permits the formation of an endothelial layer on the inside surface of the stent may therefore be particularly biocompatible, resulting in less trauma at the point of application, fewer side effects, and/or longer term device viability.

Medical appliances including a covering of porous or semi-porous material may be configured to inhibit or reduce inflammatory responses by the body toward the tissue contacting side of the medical appliance, for example. Mechanisms such as an inflammatory response by the body toward the medical appliance may stimulate, aggravate, or encourage negative outcomes, such as neointimal hyperplasia. For example, a device configured to permit tissue ingrowth and/or the growth or attachment of endothelial cells onto the blood contacting side of the device may reduce the likelihood of negative flow characteristics and blood clotting. Similarly, a device so configured may mitigate the body's inflammatory response toward the material on, for example, the tissue or non-blood contacting side of the device. By modulating the evoked inflammatory response, negative outcomes such as the presence of bioactive inflammatory macrophages and foreign body giant cells may be reduced. This may aid in minimizing the chemical chain of responses that may encourage fibrous capsule formation surrounding the device and events stimulating neointimal hyperplasia.

Rotational spun materials, such as those described herein, may be used to comprise portions of medical appliances, such as stents, patches, grafts, and so forth. The present disclosure is applicable to any implantable medical appliance, notwithstanding any specific examples included below. In other words, though particular medical appliances, such as stents or patches, may be referenced in the disclosure and examples below, the disclosure is also analogously applicable to other medical appliances, such as those which comprise a covering or layer of polymeric material.

In some embodiments, rotational spun nanofibers (and/or microfibers) may be configured to permit interaction with nano-scale (and/or micro-scale) body structures, such as endothelial cells. Rotational spinning refers generally to processes involving the expulsion of flowable material from one or more orifices, the material forming fibers which are subsequently deposited on a collector. Examples of flowable materials include dispersions, solutions, suspensions, liquids, molten or semi-molten material, and other fluid or semi-fluid materials. In some embodiments, the rotational spinning processes are completed in the absence of an electric field.

For example, one embodiment of a rotational spinning process comprises loading a polymer solution or dispersion into a cup or spinneret configured with orifices on the outside circumference of the spinneret. The spinneret is then rotated, causing (through a combination of centrifugal and hydrostatic forces, for example) the flowable material to be expelled from the orifices. The material may then form a “jet” or “stream” extending from the orifice, with drag forces tending to cause the stream of material to elongate into a small diameter fiber. The fibers may then be deposited on a collection apparatus. Exemplary methods and systems for rotational spinning can be found in U.S. Patent Publication No. US2009/0280325, titled “Methods and Apparatuses for Making Superfine Fibers,” which is herein incorporated by reference in its entirety.

Rotational spinning may be configured to create mats, tubes, or other structures comprised of elongate fibers, including nanofibers (i.e. fibers which are smaller than one micron in diameter) or microfibers (i.e. fibers which are between one micron and one millimeter in diameter). In some instances the fibers may be randomly disposed, while in other embodiments the alignment or orientation of the fibers may be somewhat controlled or follow a general trend or pattern. Regardless of any pattern or degree of fiber alignment, as the fibers are deposited on a collector or on previously deposited fibers; the fibers are not woven, but rather serially deposited on the collector or other fibers. Because rotational spinning may be configured to create a variety of structures, as used herein, the terms “mat” or “non-woven mat or material” is intended to be broadly construed as referring to any such rotational spun structure, including tubes, spheres, and so on.

The present disclosure relates to medical appliances which may have, in certain embodiments, metal scaffolding covered with at least one layer of rotational spun material, such as rotational spun polytetrafluoroethylene (PTFE). Additionally, the present disclosure relates to medical appliances formed of rotational spun materials which may not have scaffolding structures or have scaffolding structures which are not made of metal. It will be appreciated that, though particular structures and coverings are described below, any feature of the scaffolding or covering described below may be combined with any other disclosed feature without departing from the scope of the current disclosure.

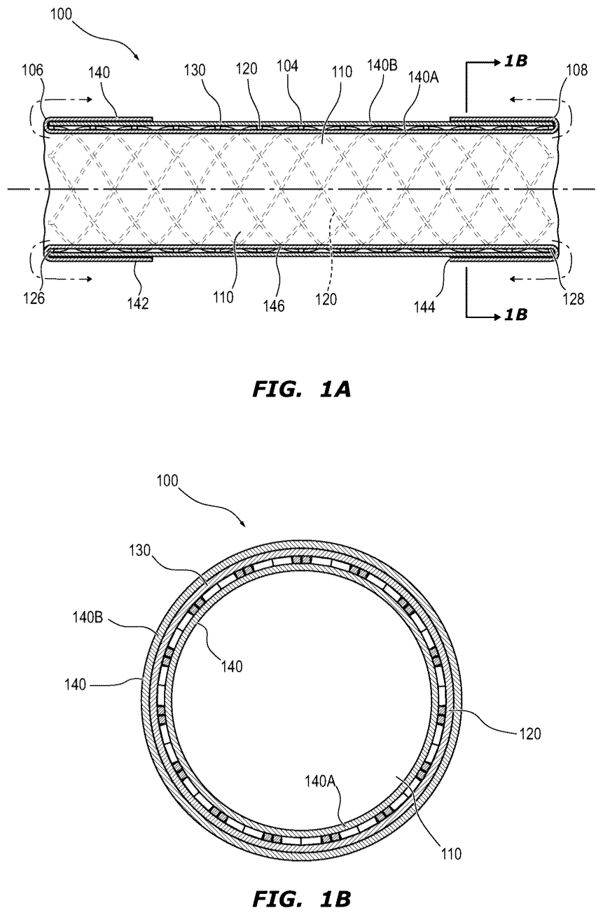

FIGS. 1A-B illustrate a covered stent graft 100 and method of manufacture, and will be described in tandem. FIG. 1A illustrates is a perspective view of a covered stent graft 100, according to embodiments described herein. FIG. 1B illustrates a cross sectional view of the covered stent graft 100 of FIG. 1A, according to embodiments described herein.

In the illustrated embodiment, stent graft 100 can include a scaffolding structure 120 and a cover comprising one or more layers (e.g., folding layer 140 and tie layer 130). In other embodiments, a stent covering may have more or fewer layers than the illustrated embodiment, including embodiments with only one covering layer. Again, disclosure recited herein with respect to specific medical appliances, such as stents, may also be applicable to other medical appliances.

Stent graft 100 can include a midbody 104 and first and second terminal ends 106, 108. At terminal ends 106, 108 the graft or cover portion of the stent graft 100 can include one or more continuous layers of material. Folding layer 140 and tie layer 130 can similarly include midbody portions, and first and second terminal ends.

In some cases, stent graft 100 can be manufactured by an everting process. For instance, as seen in FIG. 1A, stent graft 100 can be formed by first disposing the covering portion, or folding layer 140, within lumen 110 of scaffolding structure 120. Folding layer 140 can be a tube-like structure, or thin-cylinder layer, placed concentrically within scaffolding structure 120. Longitudinal ends 142, 144 can initially extend past terminal ends 126, 128 of the scaffolding structure 120.

A tie layer 130 can then be disposed concentrically exterior to the scaffolding structure 120. Folding layer 140 can then be everted, or folded outwardly, to cover tie layer 130 and scaffolding structure 120. For example, in embodiments, longitudinal ends 142, 144 of folding layer 140 can be everted outwardly to become an exterior circumferential surface of stent graft 102.

As seen, in embodiments, longitudinal ends 142, 144 can be everted over terminal ends 126, 128 of the stent scaffold structure 120. Longitudinal ends 142, 144 can be everted over terminal ends 126, 128 and tie layer 130. Longitudinal ends 142, 144 can rest on an exterior circumferential surface of tie layer 130. After longitudinal ends 142, 144 have been everted, in embodiments, the various layers and materials of stent graft 100 can be bonded together (e.g., through sintering or a similar process).

As seen, a midbody 146 of the folding layer 140 can form an interior, or inner portion 140A of the covered stent graft 102. Longitudinal ends 142, 144 can form an outer portion 140B of the covered stent graft 100. In embodiments, longitudinal ends 142, 144 can extend a distance along the outer circumferential surface of the stent graft 100. For instance, in some cases, longitudinal ends 142, 144 can extend the entire length of the stent graft 100, such that an entire outer circumferential surface of the stent graft 100 is formed from folding layer 140. In alternate embodiments, longitudinal ends 142, 144 may extend a shorter distance (as seen in FIG. 1A). In some cases, longitudinal ends 142, 144 can extend and contact each other, and may be joined by a butt-joint, overlap, use of adhesive, etc.

In some embodiments tie layer 130 can contribute specific material properties to the stent graft 102, e.g., such as bonding outer portion 140B and inner portion 140A of folding layer 140. In embodiments the tie layer 130 may further be configured to provide certain properties to the stent 102 as a whole, such as stiffness or tensile strength. The tie layer 130 may thus be configured as a reinforcing layer.

In embodiments, terminal ends 106, 108 of stent graft 102 can be scalloped ends, which may be configured to reduce infolding of the stent cover at the ends. For example, in some instances, a stent may have a larger diameter than a vessel in which it is deployed. Thus, the vessel may partially compress the stent radially. In some instances this radial compression may create folds or wrinkles in flat cut stent covers. These folds may then impede blood flow or lead to clotting within the vessel. Scalloped ends may reduce the occurrence of infolding at the end of a radially compressed stent. It is within the scope of this disclosure to use either type of end on any end of any stent.

Folding layer 140 and/or tie layer 130 can be composed of membranes composed of rotational spun mats, which may have a microstructure composed of many fibers crossing each other at various and random points. A rotational spinning process may control the thickness of this structure and thereby, the relative permeability of the mat. As more and more fibers are rotational spun onto a mat, the mat may both increase in thickness and decrease in permeability (due to successive layers of strands occluding the pores and openings of layers below).

Mats produced in connection with the present disclosure may be described by three general parameters: percent porosity, mat thickness, and fiber diameter. Each of these parameters may impact the nature of the mat, including the tendency of the mat to permit tissue ingrowth and/or endothelial attachment or the tendency of the mat to resist tissue ingrowth or endothelial attachment. Each of these parameters may be optimized with respect to each other to create a mat having particular characteristics.

Percent porosity refers to the percent of open space to closed space (or space filled by fibers) in a fiber mat. Thus, in some cases, the more open the mat is, the higher the percent porosity measurement. In some instances, percent porosity may be determined by first obtaining an image, such as an SEM, of a rotational spun material. The image may then be converted to a “binary image,” or an image showing only black and white portions, for example. The binary image may then be analyzed and the percent porosity determined by comparing the relative numbers of each type of binary pixel. For example, an image may be converted to a black and white image wherein black portions represent gaps or holes in the rotational spun mat while white portions represent the fibers of the mat. Percent porosity may then be determined by dividing the number of black pixels by the number of total pixels in the image. In some instances, a code or script may be configured to make these analyses and calculations.

In embodiments, percent porosities from about 30% to about 80% may be configured to permit tissue ingrowth into the layer and/or permit endothelial growth or attachment on the layer, including mats of about 40% to about 60%, mats of about 45% to about 50%, or mats of about 50% porosity. Less open layers may be configured to resist such ingrowth and/or attachment. Because the fibers comprising the mat are deposited in successive layers, the second parameter, mat thickness, may be related to porosity. In other words, the thicker the mat, the more layers of fibers, and the less porous the mat may be. In some embodiments, mats from about 20 micrometers to about 100 micrometers may be configured for use in connection with the present disclosure, including mats from about 40 micrometers to about 80 micrometers. Finally, the third parameter, fiber diameter, may be a measurement of the average fiber diameter of a sample in some instances. In some embodiments fiber diameters from about 50 nanometers to about 3 micrometers may be used in connection with the present disclosure. Notwithstanding these or other specific ranges included herein, it is within the scope of this disclosure to configure a mat with any combination of values for the given parameters.

In some embodiments the “average pore size” of the mat may be used as an alternate or additional measurement of the properties of the mat. The complex and random microstructure of rotational spun mats presents a challenge to the direct measurement of the average pore size of the mat. Average pore size can be indirectly determined by measuring the permeability of the mat to fluids using known testing techniques and instruments. Once the permeability is determined, that measurement may be used to determine an “effective” pore size of the rotational spun mat. As used herein, the “pore size” of a rotational spun mat refers to the pore size of a membrane which corresponds to the permeability of the rotational spun mat when measured using ASTM standard F316 for the permeability measurement. This standard is described in ASTM publication F316 “Standard Test Methods for Pore Size Characteristics of Membrane Filters by Bubble Point and Mean Flow Pore Test,” which is incorporated herein by reference. In some instances this test can be used as a quality control after configuring a mat based on the three parameters (percent porosity, thickness, and fiber diameter) discussed above.

In some applications it may be desirable to create a medical appliance such as stent graft 100 with an outer portion 140B or layer which is substantially impermeable. Such a layer may decrease the incidence of lumen tissue surrounding the stent growing into or attaching to the stent. This may be desirable in applications where the stent is used to treat stenosis or other occlusions; an impermeable outer layer may prevent tissue from growing into or through the material toward or into the lumen of the stent and reblocking or restricting the body lumen. In some embodiments a substantially impermeable outer layer may be produced by using rotational spun mats with a percent porosity from about 0% to about 50%, including about 25%; a thickness from about 20 micrometers to about 100 micrometers, including from about 40 micrometers to about 80 micrometers; and fiber diameters from about 50 nanometers to about 3 micrometers.

Additionally, or alternatively, a substantially impermeable mat may have an average pore size of about 0 microns to about 1.5 microns. In other embodiments, the impermeable layer may have an average pore size of less than about 0.5 micron. In yet other embodiments, the impermeable layer may have an average pore size of less than about 1 micron. In some embodiments, the impermeable layer may be a layer disposed inside of outer portion 140B, such as a tie layer, an intermediate layer, or an inner layer or inner portion.

In one example, a medical appliance such as stent graft 102 may be covered with a rotational spun PTFE outer portion 140B. The outer portion may be configured to be substantially impermeable to tissue ingrowth and/or attachment. In other embodiments the impermeability of the stent may be provided by a tie layer disposed beneath the outer portion. For example, a substantially impermeable layer may be formed of FEP which is applied, for example, as a film or dip coating between rotational spun layers of PTFE. Furthermore, FEP may be rotational spun with a small average pore size to create a substantially impermeable layer. In some embodiments both the tie layer and the exterior layer may be configured to be substantially impermeable. In some embodiments, the tie layer may comprise an extruded sheet of material applied to the stent graft. The tie layer may be non-fibrous.

Dip coatings may be applied by dipping a portion of a layer or construct in a polymer dispersion. For example, a PTFE layer (e.g., such as folding layer 140) may be dip coated on a construct by adding 20 ml of water to 50 ml of a 60 wt % PTFE dispersion to thin the dispersion. A fiber mat may then dipped in the solution to coat the mat. The dip coat may then sintered at 385 degrees C. for 15 minutes. Other concentrations of PTFE dispersions for dip coatings are also within the scope of this disclosure.

Further, an FEP layer may be dip coated on a construct by adding 20 ml of water to 50 ml of a 55 wt % dispersion to thin the dispersion. A fiber mat may then dipped in the solution to coat the mat. The dip coat may then cooked, for example, at 325 degrees C. for 15 minutes. Other concentrations of FEP dispersions for dip coatings are also within the scope of this disclosure. Additionally, polymer dispersions may be sprayed or otherwise applied onto a surface (such as a fiber mat) to coat the surface. Such coatings may be heat treated after application.

In some embodiments, more or less water, for example from about 10 ml to about 50 ml, may be added to similar amounts and concentrations of the dip dispersions above to thin the dispersions. Additionally, substances other than, or in addition to, water may be used to thin a dispersion for dip coating. For example, a surfactant or a solvent may be used. In some such cases the surfactant or solvent may later be removed from the construct, including embodiments where it is allowed to evaporate when the coat is sintered or cooked. Alcohols, glycols, ethers, and so forth may be so utilized.

In some embodiments it may be desirable to create a medical appliance such as stent graft 100 with an exterior layer, or outer portion 140B, which is more porous. A porous exterior layer may permit healing and the integration of the prosthesis into the body. For instance, tissue of the surrounding lumen may grow into the porous outer diameter or attach to the outer diameter layer. This tissue ingrowth may permit, modulate, and/or influence healing at the therapy site. In some embodiments a porous folding layer 140 may be formed of rotational spun PTFE.

In certain embodiments a relatively porous tie layer may be desirable. This layer may or may not be used in conjunction with a substantially impermeable exterior layer. A relatively porous inner layer may permit tissue ingrowth and/or endothelial attachment or growth on the inside diameter of the stent 102 which may be desirable for any combination of the following: healing, biocompatibility, prevention of thrombosis, and/or reducing turbulent blood flow within the stent. In some embodiments the inner layer may be comprised of a mat, such as a rotational spun PTFE mat, having a percent porosity of about 40% to about 80%, including about 50%; a thickness of about 20 micrometers to about 100 micrometers, including from about 40 micrometers to about 80 micrometers; and fiber diameters from about 50 nanometers to about 3 micrometers.

Additionally or alternatively the mat may be comprised of a rotational spun mat, such as PTFE, with an average pore size of about 1 micron to about 12 microns, such as from about 2 microns to about 8 microns, or from about 3 microns to about 5 microns, or alternatively from about 3.5 microns to about 4.5 microns.

As seen in FIG. 1B, tie layer 130 is shown above the “level” of the scaffolding structure 120, however, tie layer 130 may be above, at the same level, or below the scaffolding structure 120, in some embodiments. Further, as shown in FIG. 1B, each layer of the covering may be disposed so that there are no voids between layers.

In some embodiments, expanded PTFE (ePTFE) may be configured as a reinforcing layer (e.g., the tie layer may be a reinforcing layer). ePTFE may be anisotropic, having differing properties in differing directions. For example, ePTFE may tend to resist creep in the direction the ePTFE membrane was expanded. A reinforcing layer of ePTFE may be oriented to increase strength, resist creep, or impart other properties in a particular direction. ePTFE may be oriented such that the expanded direction is aligned with an axial direction of a medical device, a transverse direction, a radial direction, at any angle to any of these directions, and so forth. Similarly, multiple layers of ePTFE may be disposed to increase strength, resist creep, or impart other properties in multiple directions. The reinforcing layer may or may not be impermeable.

Additionally, in embodiments where both the inner portion 140A and outer portion 140B are porous in nature, the tie layer 130 may be configured to create an impermeable layer between the two porous layers. In such embodiments the stent may permit tissue ingrowth, tissue attachment and/or healing on both the inner and outer surfaces of the stent while still preventing tissue outside of the stent from growing into the lumen and occluding the lumen. Thus, tie layers may be configured to create a mid-layer portion of a construct, the tie-layer configured to inhibit tissue ingrowth into the layer or to be impervious to tissue migration into or through the layer or to substantially inhibit tissue migration.

Furthermore, the tie layer 130 may be configured to be impervious or substantially impervious to fluid migration across the tie layer 130. Specifically, constructions comprising one or more porous layers may allow fluid to cross the porous layer. In the case of a medical appliance configured to control blood flow, such as a graft, a porous layer may allow blood to leak across the layer or may allow certain smaller components of the blood to cross the layer while containing larger components, effectively filtering the blood. In some instances this filtration or ultrafiltration may allow components such as plasma to cross the barrier while containing red blood cells, leading to seroma. Thus, a fluid impermeable tie layer may be configured to contain fluid within a medical device also comprised of porous layers. In some devices, a tie layer may be both fluid impermeable and impervious to tissue ingrowth, or may be configured with either of these properties independent of the other. Constructs wherein any layer (other than, or in addition to a tie layer) is configured to be fluid impermeable and/or impervious to tissue ingrowth are also within the scope of this disclosure. Thus, disclosure recited herein in connection with fluid impermeable and/or tissue impervious tie layers may be analogously applied to impermeable layers at various locations within a construct.

The tie layer 130 (or any impermeable/impervious layer) may include any thermoplastic and may or may not be rotational spun. In one embodiment, the tie layer may be expanded PTFE. In another it may be rotational spun PTFE. In other embodiments it may be FEP, including rotational spun FEP and FEP applied as a film or dip coating. Furthermore, the tie layer may include any of the following polymers or any other thermoplastic: dextran, alginates, chitosan, guar gum compounds, starch, polyvinyl pyridine compounds, cellulosic compounds, cellulose ether, hydrolyzed polyacrylamides, polyacrylates, polycarboxylates, polyvinyl alcohol, polyethylene oxide, polyethylene glycol, polyethylene imine, polyvinylpyrrolidone, polyacrylic acid, poly(methacrylic acid), poly(itaconic acid), poly(2-hydroxyethyl acrylate), poly(2-(dimethylamino) ethyl methacrylate-co-acrylamide), poly(N-isopropylacrylamide), poly(2-acrylamido-2-methyl-I-propanesulfonic acid), poly (methoxyethylene), poly(vinyl alcohol), poly(vinyl alcohol) 12% acetyl, poly(2,4-dimethyl-6-triazinylethylene), poly(3morpholinylethylene), poly(N-I,2,4-triazolyethylene), poly(vinyl sulfoxide), poly(vinyl amine), poly(N-vinyl pyrrolidone-co-vinyl acetate), poly(g-glutamic acid), poly(Npropanoyliminoethylene), poly(4-amino-sulfo-aniline), poly [N-(p-sulphophenyl)amino-3-hydroxymethyl-1,4phenyleneimino-I,4-phenylene], isopropyl cellulose, hydroxyethyl, hydroxylpropyl cellulose, cellulose acetate, cellulose nitrate, alginic ammonium salts, i-carrageenan, N-[(3′-hydroxy-2′,3′-dicarboxy)ethyl]chitosan, konjac glocomannan, pullulan, xanthan gum, poly(allyammonium chloride), poly(allyammonium phosphate), poly(diallydimethylammonium chloride), poly(benzyltrimethylammonium chloride), poly(dimethyldodecyl (2-acrylamidoethyly) ammonium bromide), poly(4-N-butylpyridiniumethylene iodine), poly(2-N-methylpridiniummethylene iodine), poly(N methylpryidinium-2,5-diylethenylene), polyethylene glycol polymers and copolymers, cellulose ethyl ether, cellulose ethyl hydroxyethyl ether, cellulose methyl hydroxyethyl ether, poly(I-glycerol methacrylate), poly(2-ethyl-2-oxazoline), poly(2-hydroxyethyl methacrylate/methacrylic acid) 90:10, poly(2-hydroxypropyl methacrylate), poly(2-methacryloxyethyltrimethylammonium bromide), poly(2-vinyl1-methylpyridinium bromide), poly(2-vinylpyridine N-oxide), poly(2-vinylpyridine), poly(3-chloro-2-hydroxypropyl 2-methacryloxyethyldimethylammonium chloride), poly(4vinylpyridine N-oxide), poly(4-vinylpyridine), poly (acrylamide/2-methacryloxyethyltrimethylammonium bromide) 80:20, poly(acrylamide/acrylic acid), poly(allylamine hydrochloride), poly(butadiene/maleic acid), poly(diallyldimethylammonium chloride), poly(ethyl acrylate/acrylic acid), poly(ethylene glycol) bis(2-aminoethyl), poly (ethylene glycol) monomethyl ether, poly(ethylene glycol) bisphenol A diglycidyl ether adduct, poly(ethylene oxide-bpropylene oxide), poly(ethylene/acrylic acid) 92:8, poly(llysine hydrobromide), poly(I-lysine hydrobromide), poly (maleic acid), poly(n-butyl acrylate/2methacryloxyethyltrimethylammonium bromide), poly(Niso-propylacrylamide), poly(N-vinylpyrrolidone/2dimethylaminoethyl methacrylate), dimethyl sulfatequaternary, poly(N-vinylpyrrolidone/vinyl acetate), poly(oxyethylene) sorbitan monolaurate (Tween 20®), poly (styrenesulfonic acid), poly(vinyl alcohol), N-methyl-4 (4′formylstyryl)pyridinium, methosulfate acetal, poly(vinyl methyl ether), poly(vinylamine) hydrochloride, poly(vinylphosphonic acid), poly(vinylsulfonic acid) sodium salt, and polyaniline.

Furthermore, the tie layer may include elastomers, silicones, fluoroelastomers, polyamides, and/or polyurethanes.

Regardless of the material, tie layer 130 may or may not be rotational spun. Additionally, the tie layer 130 may be fibrous or non-fibrous, such as a tie layer formed of an extruded material, a dip coating, a spray coating, a film applied to the construct, and so forth.

Further, in certain embodiments the stent graft 100 may include two or more tie layers. The tie layer 130 may be formed in any manner known in the art and attached to the inner portion 140A and/or outer portion 140B in any manner known in the art. For example, the tie layer 130 can include a sheet of material which is wrapped around the inner portion 140A or a tube of material which is slipped over the inner portion 140A which is then heat shrunk or otherwise bonded to the inner portion 140A and outer portion 140B. Further, in embodiments where the tie layer is rotational spun, it may be rotational spun directly onto the inner portion 140A, the scaffolding, or both.

In some instances the tie layer 130 may be melted after the stent graft 100 is constructed to bond the tie layer 130 to adjacent layers of the stent covering.

Furthermore, tie layer(s) may be configured to change the overall properties of the medical appliance. For example, in some instances a cover or construct comprised solely of rotational spun PTFE (of the desired pore size) may not have desired tensile or burst strength. A tie layer comprised of a relatively stronger material may be used to reinforce the PTFE inner layer, the PTFE outer layer, or both. For example, in some instances FEP layers may be used to increase the material strength of the cover. Again, as discussed above, the tie layer may also be configured as a portion of the construct configured to be impervious to tissue ingrowth or migration.

Further, one or more layers of rotational spun PTFE may be used in connection with a scaffolding structure other than that shown herein. In other words, the disclosure above relating to covers, layers, tie layers, and related components is applicable to any type of scaffolding structure as well as to stents or grafts with no separate scaffolding structure at all.

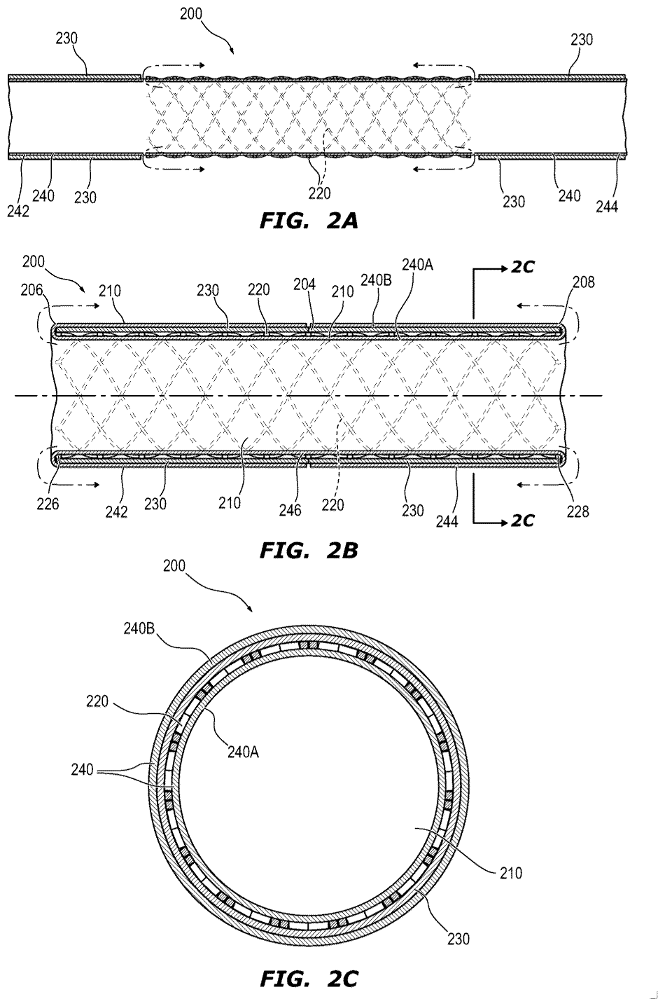

FIGS. 2A-C, 3A-B, 4A-B, and 5A-B illustrate embodiments of a covered stent graft 200, 300, 400, 500 that resemble the covered stent graft 100 described with respect to FIGS. 1A-B, in certain respects. Accordingly, like features are designated with like reference numerals, with the leading digit incremented to “2-5”. For example, the embodiment depicted in FIGS. 2A-C includes a covered stent graft 200 that may, in some respects, resemble covered stent graft 100 of FIGS. 1A-B. Relevant disclosure set forth above regarding similarly identified features thus may not be repeated hereafter. Moreover, specific features of the covered stent grafts 200, 300, 400, 500 of FIGS. 2A-C, 3A-B, 4A-B, and 5A-B, and related components shown in FIGS. 2A-C, 3A-B, 4A-B, and 5A-B, may not be shown or identified by a reference numeral in the drawings or specifically discussed in the written description that follows. However, such features may clearly be the same, or substantially the same, as features depicted in other embodiments and/or described with respect to such embodiments.

Accordingly, the relevant descriptions of such features apply equally to the features of the embodiments of the covered stent grafts 200, 300, 400, 500 and related components depicted in FIGS. 2A-C, 3A-B, 4A-B, and 5A-B. Any suitable combination of the features, and variations of the same, described with respect to the covered stent graft 100 and related components illustrated in FIGS. 1A-B can be employed with the embodiments of covered stent grafts 200, 300, 400, 500 and related components of FIGS. 2A-C, 3A-B, 4A-B, and 5A-B, and vice versa. This pattern of disclosure applies equally to further embodiments depicted in subsequent figures and described hereafter, wherein the leading digits may be further incremented.

FIGS. 2A-C, illustrate a second embodiment of a covered stent graft 200, and will be described in tandem. FIG. 2A is a lateral, cutaway view of an initial phase of a method of manufacturing a covered stent graft 200, according to embodiments described herein. FIG. 2B is a lateral, cutaway view of a manufactured covered stent graft 200 of FIG. 2A, according to embodiments described herein. FIG. 2C is a cross-sectional view of the covered stent graft 200 of FIG. 2B taken through line 20-20, according to embodiments described herein.

In the illustrated embodiments, a completed stent graft 200 can include a midbody 204 and first and second terminal ends 206, 208. At terminal ends 206, 208 the graft or cover portion of the stent graft 202 can include one or more continuous layers of material. Folding layer 240 and tie layer 230 can similarly include midbody portions, and first and second terminal ends.

In some cases, stent graft 202 can be manufactured by an everting process. For instance, as seen in FIG. 2A, stent graft 202 can be formed by first disposing the folding layer 240 within scaffolding structure 220. After, material for a tie layer 230 can be deposited onto longitudinal ends 242, 244 of folding layer 240. Folding layer 240 can then be everted, or folded outwardly, to cover scaffolding structure 220. For example, in embodiments, longitudinal ends 242, 244 of folding layer 240 can be everted outwardly to become an exterior circumferential surface of stent graft 202. In embodiments, longitudinal ends 242, 244 can be everted over terminal ends 226, 228 of the stent scaffolding structure 220.

In embodiments, depositing the material for tie layer 230 onto the outer circumferential surface of longitudinal ends 242, 244 can include processes such as spraying, wrapping, extruding, etc. The material for tie layer 230 can be deposited via more than one method. In some cases, the material for tie layer 230 can be deposited directly onto the scaffolding structure 220 and/or longitudinal ends 242, 244 in tandem.

In embodiments, as folding layer 240 is everted over terminal ends 206, 208 of covered stent graft 202, the material, or tie layer 230, can become disposed on the outer circumferential surface of the scaffolding structure 220. Otherwise stated, the material, or tie layer 230 disposed on longitudinal ends 242, 244 can become an intermediate, or tie layer, of the stent graft 220, after folding layer 240 is everted.

As illustrated, in some embodiments, midbody 246 of the folding layer 240 can form an interior, or inner portion 240A, defining the inner lumen 210 of the covered stent graft 200, while longitudinal ends 242, 244 can form an outer portion 240B of the covered stent graft 202.

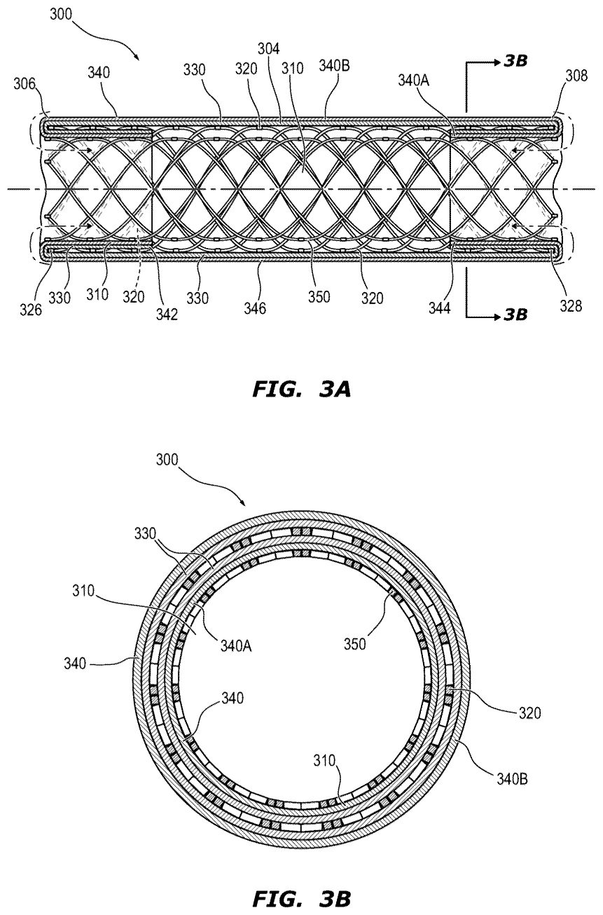

FIGS. 3A-B, illustrate a third embodiment of a covered stent graft 300, and will be described in tandem. FIG. 3A is a perspective view of a method of manufacturing a covered stent graft 300, according to embodiments described herein. FIG. 3B is a cross-sectional view of the covered stent graft 300 of FIG. 3A taken through line 3B-3B, according to embodiments described herein. Stent graft 300 can include a midbody 304 and first and second terminal ends 306, 308. At terminal ends 306, 308 the graft or cover portion of the stent graft 300 can include one or more continuous layers of material. Folding layer 340 and tie layer 330 can similarly include midbody portions, and first and second terminal ends.

In some cases, stent graft 300 can be manufactured by an inverting process involving two stent scaffolding structures. For instance, as seen in FIG. 3A, stent graft 300 can be formed by first disposing the folding layer 340 and/or tie layer 330 concentrically exterior of a first scaffolding structure 320.

As seen, in some embodiments, folding layer 340 and tie layer 330 can be of same or similar lengths. In other embodiments, the tie layer 330 may be of a shorter distance than the folding layer 340. In some cases, folding layer 340 and tie layer 330 can be pre-bonded, in others, the layers can be separate.

After placement, folding layer 340 (and/or material of tie layer 330) can be inverted, or folded inwardly, to cover first scaffolding structure 320. For example, in embodiments, longitudinal ends 342, 344 of folding layer 340 (and/or material of tie layer 330) can be inverted inwardly to be disposed against an inner circumferential surface of stent graft 300. In embodiments, longitudinal ends 342, 344 can be inverted over terminal ends 326, 328 of scaffolding structure 320.

In embodiments, as folding layer 340 is inverted over terminal ends 326, 328 of scaffolding structure 320, the material of tie layer 330 can become disposed on both an inner and outer circumferential surface of the scaffolding structure 320 (as seen in FIG. 3A). Otherwise stated, tie layer 330 can become an intermediate, or tie layer, of the stent graft 320, after folding layer 340 is inverted.

As seen, midbody 346 of the folding layer 340 can form an exterior, or outer portion 302B of the covered stent graft 302, while longitudinal ends 342, 344 can form an inner portion 340A of the covered stent graft 302. In embodiments, longitudinal ends 342, 344 can extend a short distance, or an entirety of the inner circumferential surface of the first scaffolding structure 320.

After folding layer 340 is inverted, a second scaffolding structure 350 can be disposed against an interior circumferential surface of inverted longitudinal ends 342, 344. Thus, second scaffolding structure 350 can define lumen 310, or form the interior of covered stent graft 300.

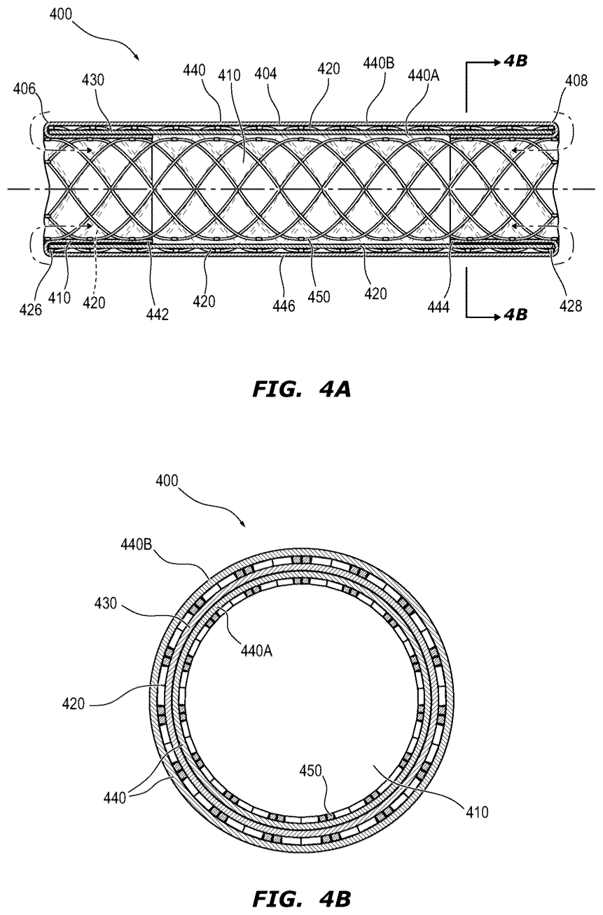

FIGS. 4A-B, illustrate a fourth embodiment of a covered stent graft 400, and will be described in tandem. FIG. 4A is a perspective view of a method of manufacturing a covered stent graft 400, according to embodiments described herein. FIG. 4B is a cross-sectional view of the covered stent graft 400 of FIG. 4A taken through line 4B-4B, according to embodiments described herein. Stent graft 400 can include a midbody 404 and first and second terminal ends 406, 408. At terminal ends 406, 408 the graft or cover portion of the stent graft 402 can include one or more continuous layers of material. Folding layer 440 and tie layer 430 can similarly include midbody portions, and first and second terminal ends.

In some cases, stent graft 400 can be manufactured by an inverting process involving two stent scaffolding structures. For instance, as seen in FIG. 4A, stent graft 400 can be formed by disposing the folding layer 440 concentrically exterior of a first scaffolding structure 420.

Folding layer 440 can then be inverted, or folded inwardly, to cover first scaffolding structure 420. For example, in embodiments, longitudinal ends 442, 444 of folding layer 440 can be inverted inwardly to be disposed against an inner circumferential surface of stent scaffolding structure 420 (and/or tie layer 430 as seen in FIG. 4B). In embodiments, longitudinal ends 442, 444 can be inverted over terminal ends 426, 428 of scaffolding structure 420.

In embodiments, prior to inverting, a second material or tie layer 430, can be disposed on or against an inner (or outer) circumferential surface of first scaffolding structure 420. Otherwise stated, in some cases, tie layer 430 can be disposed concentrically interior to the first scaffolding structure 420. In embodiments, as folding layer 440 is inverted over terminal ends 426, 428 of scaffolding structure 420, the material, or tie layer 430, can become disposed between the scaffolding structure 420 and longitudinal ends 442, 444. Otherwise stated, the material, or tie layer 430 can become an intermediate, or tie layer, of the stent graft 420, after folding layer 440 is inverted.

As seen, midbody 446 of the folding layer 440 can form an exterior, or outer portion 402B of the covered stent graft 402, while longitudinal ends 442, 444 can form an inner portion 440A of the covered stent graft 400. Longitudinal ends 442, 444 can extend any distance longitudinally along the inner circumferential surface of the stent graft 400.

After folding layer 440 is inverted, a second scaffolding structure 450 can be disposed against an inner circumferential surface of inverted longitudinal ends 442, 444. Thus, second scaffolding structure 450 can form the interior of covered stent graft 400, and define lumen 410.

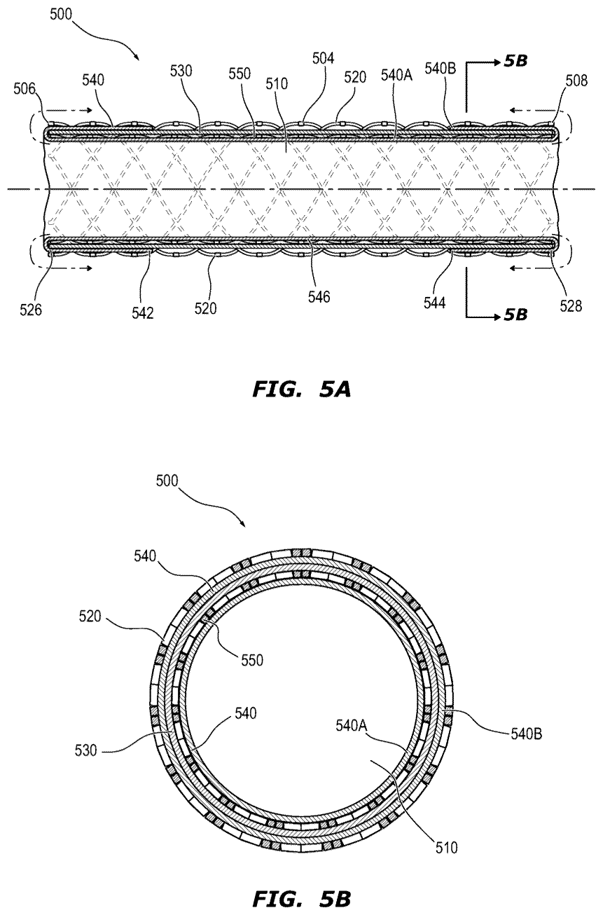

FIGS. 5A-B, illustrate a fifth embodiment of a covered stent graft 500, and will be described in tandem. FIG. 5A is a perspective view of a method of manufacturing a covered stent graft 500, according to embodiments described herein. FIG. 5B is a cross-sectional view of the covered stent graft 500 of FIG. 5A taken through line 5B-5B, according to embodiments described herein. Stent graft 500 can include a midbody 504 and first and second terminal ends 506, 508. At terminal ends 506, 508 the graft or cover portion of the stent graft 502 can include one or more continuous layers of material. Folding layer 540 and tie layer 530 can similarly include midbody portions, and first and second terminal ends.

In some cases, stent graft 500 can be manufactured by an everting process involving two stent scaffolding structures. For instance, as seen in FIG. 5A, stent graft 500 can be formed by disposing the folding layer 540 concentrically interior of a first scaffolding structure 520.

Folding layer 540 can then be everted, or folded outwardly, to cover first scaffolding structure 520. For example, in embodiments, longitudinal ends 542, 544 of folding layer 540 can be everted outwardly to be disposed exterior to an outer circumferential surface of first scaffolding structure 550. In embodiments, longitudinal ends 542, 544 can then be everted over terminal ends 526, 528 of scaffolding structure 550.

In embodiments, prior to everting, tie layer 530 (and/or second scaffolding structure 520), can be disposed on an outer circumferential surface of first scaffolding structure 550. Otherwise stated, in some cases, tie layer 530 (and/or second scaffolding structure 520) can be disposed concentrically exterior to the first scaffolding structure 550. In embodiments, as folding layer 540 is everted over terminal ends 526, 528 of scaffolding structure 550, the material, or tie layer 530, can become disposed between the scaffolding structure 520 and longitudinal ends 542, 544. Otherwise stated, the material, or tie layer 530 can become an intermediate, or tie layer, of the stent graft 500, after folding layer 540 is everted.

As seen, midbody 546 of the folding layer 540 can form an interior, or inner portion 540A of the covered stent graft 502 and define lumen 510. Longitudinal ends 542, 544 can form an outer portion 540B of the covered stent graft 500. Longitudinal ends 542, 544 can extend any distance longitudinally along the outer circumferential surface of the stent graft 500.

In some embodiments, second scaffolding structure 520 can then be disposed against an exterior circumferential surface of everted, longitudinal ends 542, 544. Thus, in some cases, second scaffolding structure 520 can form the exterior of covered stent graft 500. In alternate embodiments, second scaffolding structure 520 can be disposed exterior to the tie layer 530, and interior to everted, longitudinal ends 542, 544. In such cases, longitudinal ends 542, 544 can form the exterior of covered stent graft 500.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

References to approximations are made throughout this specification, such as by use of the term “near.” For each such reference, it is to be understood that, in some embodiments, the value, feature, or characteristic may be specified without approximation. For example, where qualifiers such as “near” and “approximately” are used, these terms include within their scope the qualified words in the absence of their qualifiers. For example, where the term “approximately aligned” is recited with respect to a feature, it is understood that in further embodiments, the feature can have a precisely aligned configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A medical appliance, comprising:
a stent; and
a first conduit comprising:
a body portion disposed against an inner circumferential surface of the stent;
a first bend everting over a first longitudinal end of the stent; and
a first end portion defining an outer circumferential surface of the medical appliance.

2. The medical appliance of claim 1, wherein the first conduit further comprises:
a second bend everting over a second longitudinal end of the stent; and
a second end portion defining an outer circumferential surface of the medical appliance.

3. The medical appliance of claim 1, wherein an inner circumferential surface of the body portion defines a lumen of the medical appliance.

4. The medical appliance of claim 1, wherein a second conduit is disposed against an outer circumferential surface of the stent.

5. The medical appliance of claim 1, wherein an inner circumferential surface of a second conduit is disposed against an outer circumferential surface of the stent; wherein the first end portion and a second end portion of the first conduit are disposed against an outer circumferential surface of the second conduit.

6. The medical appliance of claim 1, wherein the first conduit comprises an outer layer which is impervious to cell migration.

7. The medical appliance of claim 1, wherein the first conduit comprises:
an inner layer comprising serially deposited polytetrafluoroethylene (PTFE) fibers; and
an outer layer comprising fluorinated ethylene propylene (FEP).

8. The medical appliance of claim 1, wherein the first end portion and the second end portion of the first conduit comprise an inner layer and an outer layer, wherein the body portion comprises one layer.

9. The medical appliance of claim 1, wherein the stent is a bifurcated stent.

10. A method of manufacturing a medical appliance, comprising:
disposing a first conduit within a stent;
everting a first end portion of the first conduit over a first longitudinal end of the stent;
everting a second end portion of the first conduit over a second longitudinal end of the stent;
bonding the everted first end portion to the stent; and
bonding the everted second end portion to the stent.

11. The method of claim 10, wherein the everted first end portion and everted second end portion to the stent define an outer circumferential surface of the medical appliance.

12. The method of claim 10, wherein bonding the everted first end portion and everted second end portion to the stent comprises bonding the everted first end portion and everted second end portion to one another.

13. The method of claim 10, wherein bonding the everted first end portion and everted second end portion to the stent comprises forming a butt joint or overlap between the everted first end portion and everted second end portion.

14. The method of claim 10, wherein bonding the everted first end portion and everted second end portion to the stent comprises sintering the medical appliance.

15. The method of claim 10, further comprising:
disposing a second conduit against an outer circumferential surface of the stent.

16. The method of claim 10, wherein the first conduit comprises an outer layer which is impervious to cell migration.

17. The method of claim 10, wherein the stent is a bifurcated stent.

18. A medical appliance, comprising:
a stent; and
a first conduit comprising:
a body portion disposed against an outer circumferential surface of the stent;
a first bend inverting over a first longitudinal end of the stent;
a second bend inverting over a second longitudinal end of the stent; and
a first end portion and a second end portion defining an inner circumferential surface of the medical appliance.

19. The medical appliance of claim 18, wherein an inner circumferential surface of the body portion defines a lumen of the medical appliance.

20. The medical appliance of claim 18, wherein the first conduit comprises an outer layer which is impervious to cell migration.
